(19) United States
(12) Patent Application Publication
(10) Pub. No.: US 2023/0290513 A1
(43) Pub. Date: Sep. 14, 2023

(54) METHOD FOR DETERMINING A DISEASE PROGRESSION AND SURVIVAL PROGNOSIS FOR PATIENTS WITH AMYOTROPHIC LATERAL SCLEROSIS

(71) Applicants: UNIVERSITA’ DEGLI STUDI DI PADOVA, PADOVA (IT); UNIVERSITA’ DEGLI STUDI DI TORINO, TORINO (IT); AZIENDO OSPEDALIERA UNIVERSITARIA MODENA, MODENA (IT); NEMO LAB SRL, MILANO (IT); THE MEDICAL RESEARCH, INFRASTRUCTURE AND HEALTH SERVICES FUND OF THE TEL-AVIV MEDICAL CENTER, TEL AVIV (IL); ISTITUTI CLINICI SCIENTIFICl MAUGERI, PAVIA (IT); HADASIT MEDICAL RESEARCH SERVICES AND DEVELOPMENT LTD., JERUSALEM (IL)

(72) Inventors: Barbara DI CAMILLO, PADOVA (IT); Alessandro ZANDONA', ARZERGRANDE (IT); Sebastian DABERDAKU, PADOVA (IT); Erica TAVAZZI, PADOVA (IT); Adriano CHIO', TORINO (IT); Rosario VASTA, TORINO (IT); Andrea CALVO, TORINO (IT); Cristina MOGLIA, AOSTA (IT); Federico CASALE, VOLPIANO (IT); Fabrizio D'OVIDIO, TORINO (IT); Jessica MANDRIOLI, MODENA (IT); Christian LUNETTA, CESANO BOSCONE (IT); Vivian DRORY, RISHON LEZION (IL); Gabriele MORA, MILANO (IT); Marc GOTKINE, NEVE MICHEAL (IL)

(21) Appl. No.: 18/017,196

(22) PCT Filed: Jul. 22, 2020
(86) PCT No.: PCT/IT2020/000057

§ 371 Date: Jan. 20, 2023

(51) Int. Cl. G16H 50/30; G16B 20/20; G06N 7/01

(52) U.S. Cl. CPC G16H 50/30; G16B 20/20; G06N 7/01

(57) ABSTRACT

A method is described for determining a disease progression and survival prognosis, at a succession of prediction times, for patients suffering from amyotrophic lateral sclerosis (ALS). The method comprises a step of defining a set of variables associated with the onset and progression of amyotrophic lateral sclerosis, comprising a first group of variables associated with the onset of amyotrophic lateral sclerosis (comprising at least the variables “patient sex”, “disease onset age”, “disease onset site”), a second group of dynamic time variables (comprising at least the variable “time elapsed since disease onset”), a third group of dynamic functional variables (comprising at least one of the variables breathing, swallowing, communicating, walking/self-care or at least one variable of a functional progression and/or severity scale of amyotrophic lateral sclerosis), and further at least one variable associated with survival. The method further provides for encoding by means of a Dynamic Bayesian Network, using at least one trained algorithm, a plurality of probabilistic conditional dependence relationships, in which each relationship is a probabilistic conditional dependence relationship between two of the aforesaid variables. The aforesaid prediction times are defined so that each prediction time belongs to a respective time interval in which the conditional dependence relationships between the variables are stationary. The method further involves describing the Dynamic Bayesian Network, using at least one trained algorithm, by means of a corresponding graph, comprising said variables as nodes and comprising topological connections oriented between nodes corresponding to variables among which a probabilistic conditional dependence is identified. In the graph, given a node, the connections entering it show a conditional probability of the value assumed by the variable associated with such node, in a given prediction time, depending on the values assumed, in a prior prediction time, from the variables associated with the nodes from which such connections originate. The method further comprises the steps of entering, for each of the defined variables, data acquired at a given acquisition time relating to the situation of a specific patient; and calculating, by electronic processing and/or calculating means, on the basis of the Dynamic Bayesian Network and the graph, and starting from the aforesaid acquired data, the values of each of the defined variables, at one or more prediction times following the acquisition time. Finally, the method involves obtaining, in a given prediction time, disease progression prognosis results on the basis of the values of one or more of the variables of the third group calculated in such prediction time; and the survival prognosis results on the basis of the value of at least one variable associated with survival, calculated at such prediction time.

### FIELD OF APPLICATION

The present invention relates to a method for determining a disease progression and survival prognosis for patients with amyotrophic lateral sclerosis.

The general technical field of the present invention is therefore that of predictive methods, performed by means of electronic computation, used in the medical field to support predictive prognoses.

### DESCRIPTION OF BACKGROUND ART

Amyotrophic Lateral Sclerosis (ALS) is a fatal neurodegenerative disorder characterized by the degeneration of motor neurons. It causes the paralysis of all voluntary muscles, usually leading to death or respirator-dependence within 4 years from onset.

The incidence of ALS in Europe and in populations of European descent is 2.6 cases for 100.000 people per year and the prevalence is of 7/9 cases per 100.000 people; ALS rates are mainly unknown for other ethnic groups.

The phenotype of ALS is heterogeneous and eight phenotypic categories have been described.

Onset may be bulbar or spinal, affecting predominantly upper or lower motor neurons. Moreover, a variety of non-motor symptoms can accompany the paralysis, with frontotemporal dementia (FTD) being the most common. The multifaceted aetiology of the disease is reflected by the fact that only 5/10% of ALS cases are familial, with the remaining vast majority being sporadic.

More than thirty different genetic conditions have been linked to ALS, with the most notable being a hexanucleotide repeat expansion at C9orf72, which was identified as significantly associated with ALS in both familial and sporadic cases.

The progression rate and pattern can be highly variable, progressively impairing the ability to move, communicate, swallow, and breathe.

The life expectancy is shorter than three years for half of the patients, with only 10% surviving for more than 10 years.

Considering its heterogeneity, predicting the progression of ALS patients would improve prognostication and intervention timing in routine clinical practice.

Moreover, clinical trials could be more effectively designed, for example by ensuring allocation of equivalent populations to the various intervention arms of a trial.

Finally, a stratification of ALS patients by their progression or phenotype could give hints on different mechanisms acting in its pathogenesis.

However, just for its heterogeneity, predicting the progression of ALS patients is not easy, and the need of improved reliability prediction methods is felt.

In order to enhance and accelerate translational ALS research, Prize4Life and the Neurological Clinical Research Institute (NCRI) at Massachusetts General Hospital created the Pooled Resource Open-Access ALS Clinical Trials (PRO-ACT) platform ((Nazem Atassi, James Berry, Amy Shui, Neta Zach, Alexander Sherman, Ervin Sinani, Jason Walker, Igor Katsovskiy, David Schoenfeld, Merit Cudkowicz, et al. “The PRO-ACT database design, initial analyses, and predictive features”. Neurology, 83(19):1719{1725, 2014).

So far, several predictive models of the ALS progression have been developed on this dataset to predict the future progression of the disease (for example in: Robert Küffner, Neta Zach, Raquel Norel, Johann Hawe, David Schoenfeld, Liuxia Wang, Guang Li, Lilly Fang, Lester Mackey, Orla Hardiman, et al. “Crowdsourced analysis of clinical trial data to predict amyotrophic lateral sclerosis progression”. Nature biotechnology, 33(1):51, 2015; or in: Albert A. Taylor, Christina Fournier, Meraida Polak, Liuxia Wang, Neta Zach, Mike Keymer, Jonathan D. Glass, David L. Ennist, and Pooled Resource Open-Access ALS Clinical Trials Consortium “Predicting disease progression in amyotrophic lateral sclerosis”. Annals of clinical and translational neurology, 3(11):866{875, 2016) and to stratify the patients into meaningful subgroups (for example in: Mei-Lyn Ong, Pei Fang Tan, Joanna D Holbrook “Predicting functional decline and survival in amyotrophic lateral sclerosis”. PloS one, 12(4):e0174925, 2017, or in Robert Küffner, Neta Zach, Maya Bronfeld, Raquel Norel, Nazem Atassi, Venkat Balagurusamy, Barbara Di Camillo, Adriano Chio, Merit Cudkowicz, Donna Dillenberger, et al. “Stratification of amyotrophic lateral sclerosis patients: a crowdsourcing approach”. Scientific reports, 9(1):690, 2019).

PRO-ACT represents an invaluable resource for research studies on ALS: its large sample size guarantees high statistical power; moreover, patients participating in clinical trials have more frequent visits, allowing for a better characterization of disease progression.

Nonetheless, clinical trial population is not necessarily representative of the general ALS population: patients participating in clinical trials are generally higher functioning and more homogeneous compared to the ones from a typical tertiary care clinic setting. Furthermore, the duration of their follow-up is often limited.

For these reasons, patient data from the clinical context should be included in the development of ALS progression models in order to achieve reliable predictions for the general ALS population.

In attempting to respond to the aforesaid needs, a model for prognostic prediction at the individual patient level was recently developed, for example, based on data collected by different European ALS treatment centers: Westeneng—2018, “Prognosis for patients with amyotrophic lateral sclerosis: development and validation of a personalised prediction model”.

Even though the above mentioned models are able to predict single survival or intervention endpoints, it would be additionally useful to also model the entire disease progression over time, considering all the dynamic variables and their relationships.

The literature models currently lack this capability, since their predictions are limited to pre-defined time points. Furthermore, they merely capture the associations among the clinical variables and the outcomes, without an explicit interpretation of interactions among variables and how these might change in time, thus not fully exploiting the richness of the dynamic data.

By further exploiting the potentiality of artificial intelligence, a model able to capture and employ this dynamic nature of the data would be useful not only for allowing a continue prognosis prediction but also for generating new “in silico” patients with different characteristics. Such models could be useful, for instance, to simulate the natural evolution of the disease in groups of untreated patients with different onset sites, in order to mimic the disease progression in in silico placebo cohorts, further allowing patient stratification studies.

In light of the above, there is therefore a strong need to have methods for determining a prognosis of ALS progression and survival, which at least partially overcomes the aforesaid limits of the known methods and, in particular, which provides more accurate and reliable predictive results with respect to the aforesaid known methods, at a succession of prediction times such as to allow a prediction of the progression of the disease and its main symptoms over time.

### SUMMARY OF THE INVENTION

It is the object of the present invention to provide a method for determining a disease progression and survival prognosis, at a succession of prediction times, for patients with amyotrophic lateral sclerosis (ALS), which allows at least partially overcoming the drawbacks mentioned above with reference to the background art, and responding to the aforementioned requirements particularly felt in the technical field considered. This object is achieved by a method in accordance with claim 1.

Further embodiments of this method are defined by claims 2-18.

A further object of the present invention is to provide a method for determining a statistical classification and/or stratification of patients suffering from ALS. This method is defined by claim 19.

A further object of the present invention is to provide a method for identifying and/or weighing risk factors of amyotrophic lateral sclerosis (ALS). This method is defined by claim 20.

### BRIEF DESCRIPTION OF THE DRAWINGS

Further characteristics and advantages of the method according to the invention will be apparent from the following description of preferred embodiments, given by way of non-limiting example, with reference to the accompanying figures, in which:

FIG. 1 is a diagram comprising a graph representative of probabilistic relationships between variables associated with the onset and progression of amyotrophic lateral sclerosis, used in a first embodiment of the method according to the invention;

FIG. 2 is a diagram comprising a graph representative of probabilistic relationships between variables associated with the onset and progression of amyotrophic lateral sclerosis, used in a second embodiment of the method according to the invention;

FIGS. 3 and 4 show two respective comparison examples between the evolutions found (in data of patient sets of known clinical evolution) and the simulated evolutions of the progression over time of different symptoms of ALS, deriving from two respective method training and validation sessions, on two different known datasets;

FIG. 5 shows a graphical interface of a software application which allows carrying out the method, according to an implementation example.

### DETAILED DESCRIPTION

A method is described for determining a disease progression and survival prognosis, at a succession of prediction times, for patients suffering from amyotrophic lateral sclerosis (ALS).

The method comprises a step of defining a set of variables associated with the onset and progression of amyotrophic lateral sclerosis, comprising a first group of variables associated with the onset of amyotrophic lateral sclerosis, a second group of dynamic time variables, a third group of dynamic functional variables, and also at least one variable associated with survival.

The first group of variables associated with the onset of amyotrophic lateral sclerosis comprises at least the variables “patient sex,” “disease onset age,” “disease onset site.”

The second group of dynamic time variables comprising at least the variable “time elapsed since disease onset.”
The third group of functional dynamic variables associated with disease effects comprises at least one of the variables breathing, swallowing, communicating, walking/self-care, or at least one variable of a functional amyotrophic lateral sclerosis progression and/or severity scale.

The method further provides for encoding by means of a Dynamic Bayesian Network, using at least one trained algorithm, a plurality of probabilistic conditional dependence relationships, in which each relationship is a probabilistic conditional dependence relationship between two of the aforesaid variables.

The method further comprises the steps of defining the aforesaid prediction times, so that each prediction time belongs to a respective time interval in which the conditional dependence relationships between the variables are stationary, that is, time-invariant or homogeneous; and defining a time variable representative of the prediction time.

The method further involves describing the Dynamic Bayesian Network, using at least one trained algorithm, by means of a corresponding graph, comprising the aforesaid variables as nodes and comprising topological connections oriented between nodes corresponding to variables among which a probabilistic conditional dependence is identified.

In the aforesaid graph, given a node, the connections entering therein represent a conditional probability of the value assumed by the variable associated with such node depending on the values of the variables associated with the nodes from which such connections originate.

At least one of the aforesaid connections is associated with a conditional probability of the value of the variable in which the connection is entering, in a given prediction time, depending on the value of the variable from which the connection is leaving in a previous prediction time.

Furthermore, for the nodes associated with the functional dynamic variables belonging to the third group of variables, respective local cycle connections entering and leaving the same node are expected, adapted to describe the influence of the respective dynamic functional variable on itself over time.

The method further comprises the steps of entering, for each of the defined variables, data acquired at a given acquisition time relating to the situation of a specific patient; and calculating, by electronic processing and/or calculating means, on the basis of the aforesaid Dynamic Bayesian Network and the aforesaid graph, and starting from the aforesaid acquired data, the values of each of the defined variables, at one or more prediction times following the acquisition time.

Finally, the method involves obtaining disease progression prognosis results, in a given prediction time, on the basis of the values of one or more of the variables of the third group calculated in such prediction time; and obtaining survival prognosis results at a given prediction time on the basis of the value of at least one variable associated with survival, calculated in such prediction time.

According to an embodiment of the method, the set of variables only comprises said first group of variables associated with the onset of amyotrophic lateral, second group of dynamic time variables, third group of dynamic functional variables and a fourth group of variables comprising at least said variable associated with survival.

Such embodiment advantageously allows to maintain good quality prediction results (by virtue of the above mentioned features) with a minimum set of indispensable group of variables.

According to an embodiment of the method, the set of variables associated with the onset, progression and effects of amyotrophic lateral sclerosis further comprises a fifth set of variables comprising genetic variables representative of the presence of possible “genetic mutations.”

According to an implementation option, this fifth group of variables comprises the variables: WT, C9orf72, TARDBP, SOD1, FUS.

According to various possible implementation variants of the method, the first group of variables associated with the onset of amyotrophic lateral sclerosis further comprises one or more of the following variables: presence of “frontotemporal dementia (FTD)” and/or “body mass index (BMI) prior to disease onset,” and/or “diagnostic delay” and/or “medical center following the patient” and/or “familiality,” and/or “body mass index (BMI) at diagnosis” and/or “forced vital capacity at diagnosis (FVC).”

According to an implementation option, the second group of dynamic time variables further comprises the variable “time between consecutive visits.”

In accordance with an embodiment of the method, the third group of dynamic functional variables comprises all the variables breathing, swallowing, communicating, walking/self-care.

Note that the aforesaid variables are expressed with the nomenclature used by the well-known classification system “Milano-Torino staging system” (MITOS). Beyond the definition used, these variables unequivocally refer to the functions most severely affected by ALS, namely “breathing,” “swallowing,” “communicating,” “walking/self-care,” the latter sometimes indicated with the more generic term “movement.”

According to an implementation option, the third group of dynamic functional variables further comprises “non-invasive ventilation (NIV)” and “percutaneous endoscopic gastrostomy (PEG).”

According to another embodiment of the method, the third group of dynamic functional variables comprises at least one variable of an ALSFRS-R functional scale.

With reference to the graph describing the Dynamic Bayesian Network (DBN), according to an implementation option of the method, such graph is a direct graph.

It should be noted that the graph obtained and used in the present method, from a certain point of view, can be seen as an acyclic graph, because the same dynamic variable in two successive times (that is, in two successive “prediction times”) corresponds, in fact, to two distinct variables.

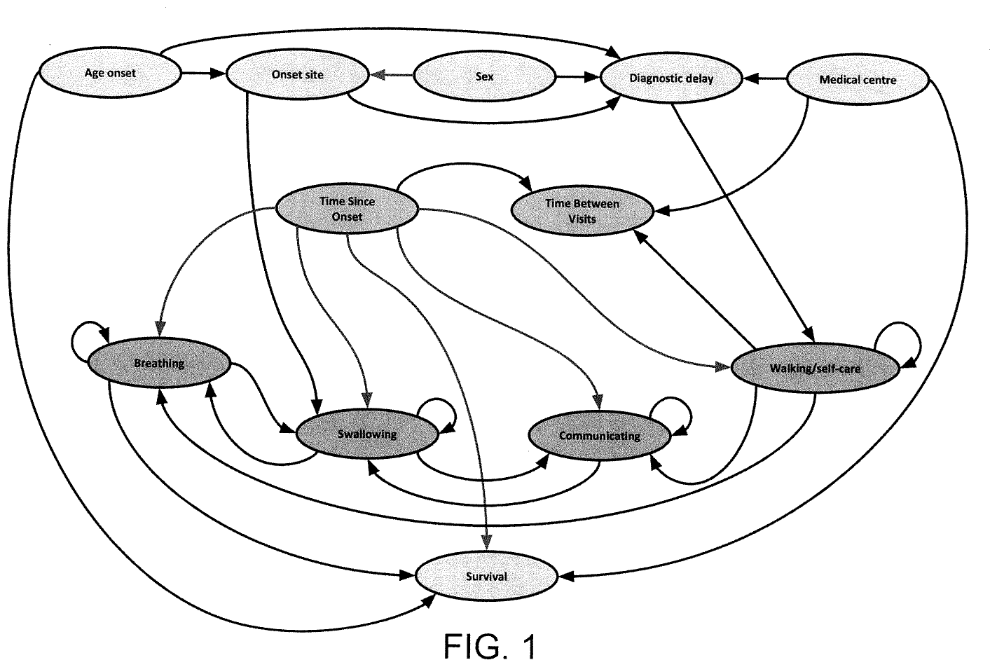

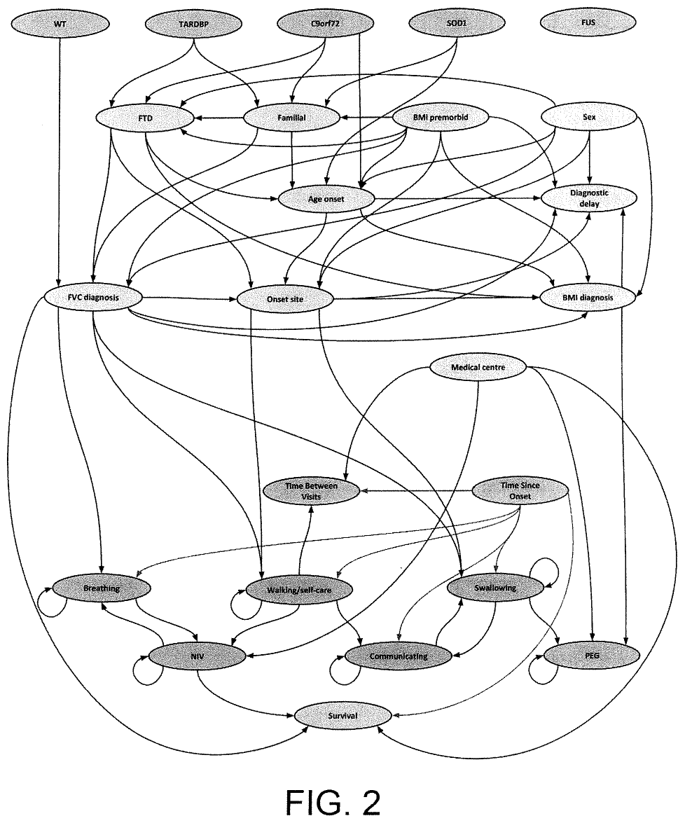

In the graphic representation used in FIGS. 1 to 2, the nodes corresponding to the same variable at two different times are made to “collapse” in a single node, while simultaneously introducing an autocycle referred to such node. Thereby an acyclic graph is shown through a representation which is no longer acyclic. As known, however, acyclic graphs can be made cyclical by adding arcs with probability equal to 0, just as an indirect graph can be made direct by adding a return arc.

Furthermore, these graphs, and the corresponding Bayesian Dynamic Networks, can also be represented differently, for example by means of tables (as will be exemplified below).

According to an embodiment, each connection of the graph is associated with a conditional probability of the value of the variable in which the connection is entering, in a given prediction time, depending on the value of the variable from which the connection is leaving in a previous prediction time.

In other words, at least one node of the graph is a child node whose value depends on the value of one or more parent nodes, and in which the respective one or more connections from the parent node(s) to the child node are associated with the conditional probabilities describing the influence of each of the parent nodes on the child node.

From another perspective, the variables of the child nodes can be seen as in turn dependent on “metavariables” which are the composition of the variables of the parent nodes.

In accordance with an embodiment of the method, the aforesaid step of describing the Dynamic Bayesian Network, by means of a corresponding graph, using at least one trained algorithm, is carried out in a preliminary training step comprising the steps of:
i) inference of the topology of the graph and
ii) learning the parameters of each conditional probability distribution, CPD, corresponding to the probability that a variable assumes a specific conditional value on each possible joint assignment of values, that is, on the possible combinations of values, of the variables in the parent nodes thereof.

According to an implementation option of the method, the aforesaid preliminary training step is carried out on the basis of one or more available experimental datasets, divided into a training set and a test set, on which machine learning and/or data mining algorithms are applied.

According to an implementation option, the training step is carried out by dividing the population disease evolution time interval into sub-intervals, within which lies the temporal stationarity hypothesis of the relationships for the dynamic functional variables of the third group and the time variable of the second group, “time elapsed since disease onset.”

According to a particular implementation example described here in more detail, the preliminary training step comprises a definition of the Bayesian Dynamic Network model, DBN, in which the DBN structure is defined using the Max-Min Hill-Climbing algorithm (MMHC) and using the Bayesian Information Criterion (BIC) parameter as the score function.

The parameters relating to the conditional probability distributions CPD are calculated using a Maximum A Posteriori (MAP) estimation for each node.

Furthermore, in this example constraints are introduced in the definition of the DBN structure to exclude clinically or biologically nonsensical relationships (as will be further exemplified below).

In accordance with an embodiment of the method, the aforesaid step of calculating the values of each of the variables defined at one or more successive times comprises iterating the following procedure: calculating the value of each of the variables corresponding to the nodes of the graph in an instant t+1 (that is, prediction time t+1) on the basis of the values of the variables associated with the respective parent nodes at the instant t (that is, prediction time t) sampling according to the probability values obtained from the conditional probability distribution inferred by the graph.

According to an embodiment of the method, the aforesaid step of obtaining disease progression prognosis results comprises predicting a temporal evolution of the dynamic functional variables of the third group.

In accordance with an embodiment, the method further comprises a step of providing and/or making available and/or displaying digital data corresponding to the prognosis and/or survival prediction results.

According to an implementation option, the method comprises the further step of providing a computerized graphical interface, configured to receive input data relating to patient variable values, relating to a specific instant in time, and to display the temporal evolution prediction results of the third group and/or survival prediction variables.

Some details about the Dynamic Bayesian Networks used in the present method are shown below for illustrative purposes.

Bayesian Networks (BNs) are descriptive models that encode the probabilistic relationships among variables. Given a multivariate dataset, the BNs build a directed acyclic graph in which each variable corresponds to a node and the influence of one node (parent) on another (child) corresponds to a directed edge. Dynamic Bayesian Networks (DBNs) are an extension of BNs well suited for describing the evolution of diseases, since they provide an explicit representation of the variable set and their inter-dependencies, as well as the means to learn not only from statistical data, but also from domain literature and expert knowledge. DBNs describe the dependencies among variables over time, with edges representing the influence of a parent variable at time step t on the child at time step t+1.

To learn the DBNs from the data, according to an implementation option, it is possible to use bnstruct (Alberto Franzin, Francesco Sambo, and Barbara Di Camillo. “bnstruct: an R package for Bayesian Network structure learning in the presence of missing data”. Bioinformatics, 33(8):12500 252, 2017), an R package that performs structure and parameter learning on discrete/categorical data even in the presence of missing values, which is a common situation in the clinical context.

An example of the processing of a DBN-based model, according to an embodiment of the method of the invention, is described below purely by way of non-limiting example.

A DBN model is developed using the Max-Min Hill-Climbing algorithm MMHC (loannis Tsamardinos, Laura E. Brown, Constantin F. Aliferis “The max-min hill-climbing Bayesian network structure learning algorithm” Machine Learning, 65(1):31-78, October 2006) with the “Bayesian Information Criterion (BIC) as score function, followed by a Maximum A Posteriori (MAP) estimation; the MMHC algorithm detects the dependencies among variables, whereas the MAP estimation weights the influence of each variable on the others.

Sense constraints are also applied to the network structure to codify the domain knowledge: clinically or biologically nonsensical relations among variables are forbidden, such as, for instance, the dependence of medical center on patient's sex.

In detail, in the learning process, the DBN model infers a set of conditional probability distributions (CPDs) for each variable; thus, DBNs are able to identify the combination of factors modulating ALS severity over its course.

Typically, DBNs are time-invariant models, which means that the dependence of the variables at time step t on the ones at the previous time step t-1 does not change in time. In the reality of clinical data this working hypothesis is not always verified.

To address this issue, the learning model has been modified, in this method, by dividing the observed disease development time framework into intervals in which the working hypothesis is verified. Considering the frequency of events, i.e., the probabilities of MITOS impairment (the already mentioned “Breathing, Swallowing, Communicating, Walking/self-care”) and tracheostomy/death, as a function of time since onset and other dynamic variables, the inflection points of the curves can be considered as timestamps of time-invariance loss. Therefore, we define time intervals (the above mentioned time intervals in which the “prediction time moments” are defined) spanning from one inflection point to the next one.

Moreover, time is used as a predictive variable, because each temporal interval defines a completely different set of conditional probabilities.

With reference to FIGS. 1 and 2, two embodiments of the method, and in particular two graphs obtained and used in the method according to the invention, will now be described in detail, by way of non-limiting example.

In both examples, the training step was carried out on the basis of the following principles.

When learning the structure of the network from the training set, the following information is provided:
1. the mandatory edges, i.e., the edges between variables that must be present in the network;
2. the possible edges, i.e., the edges that can be found during the learning phase. By default, after grouping variables in separate (disjoint) layers, variables in a given layer j can depend only on variables from layers i minor than or equal to j. Users, however, can allow or deny specific dependencies between layers.

The first graph shown in FIG. 1 was obtained on the basis of a first dataset of experimental clinical data, the details of which will be provided in a subsequent part of this description.

The mandatory edges set for the network learned on the first dataset are:
the dependency of Onset Site from Sex;
the dependencies of the variables MITOS (the already cited “Breathing, Swallowing, Communicating, Walking/self-care”) at time t from the variable Time Since Onset;
the dependencies of the variable Survival from the variable Time Since Onset.

For the network on the first dataset, the variable layering was defined as follows.
Layer 1: Sex, Age onset
Layer 2: Medical centre
Layer 3: Onset site
Layer 4: Diagnostic delay
Layer 5: MITOS variables at time (t-1)
Layer 6: Time Between Visits
Layer 7: MITOS variables at time t
Layer 8: Survival
Layer 9: Time Since Onset.

The following rules were imposed through the learning phase:
Layer 1 cannot depend on itself or any other layer.
Layer 2 cannot depend on itself or any other layer.
Layer 3 can only depend on itself and layer 1.
Layer 4 can only depend on layers 1, 2 and 3.
Layer 5 cannot depend on itself or any other layer.
Layer 6 can only depend on layer 2 and 5.
Layer 7 can depend on any other layer, except for itself and layers 6 and 8.
Layer 8 can depend on any other layer, except for itself and layers 6 and 7.
Layer 9 cannot depend on itself or any other layer.

These rules are given in matrix form in Table 1.

TABLE 1

| | L1 | L2 | L3 | L4 | L5 | L6 | L7 | L8 | L9 |
|---|---|---|---|---|---|---|---|---|---|
| L1 | 0 | 0 | 1 | 1 | 0 | 0 | 1 | 1 | 0 |
| L2 | 0 | 0 | 0 | 1 | 0 | 1 | 1 | 1 | 0 |
| L3 | 0 | 0 | 1 | 1 | 0 | 0 | 1 | 1 | 0 |
| L4 | 0 | 0 | 0 | 0 | 0 | 0 | 1 | 1 | 0 |
| L5 | 0 | 0 | 0 | 0 | 0 | 1 | 1 | 1 | 0 |
| L6 | 0 | 0 | 0 | 0 | 0 | 0 | 0 | 0 | 0 |
| L7 | 0 | 0 | 0 | 0 | 0 | 0 | 0 | 0 | 0 |
| L8 | 0 | 0 | 0 | 0 | 0 | 0 | 0 | 0 | 0 |
| L9 | 0 | 0 | 0 | 0 | 0 | 0 | 1 | 1 | 0 |

A given element A_{i,j} at row i and column j, if equal to 1 indicates that the variables of layer j can depend on those of layer i. Otherwise, if A_{i,j} is equal to 0, it means that the dependency of layer j on layer i is forbidden.

FIG. 1 reports the network obtained from the first training set.

The mandatory edges set for the network learned on the second dataset are:
the dependencies of the variables MITOS (the already cited “Breathing, Swallowing, Communicating, Walking/self-care”) at time t from the variable Time Since Onset;
the dependency of the variable Survival from the variable Time Since Onset;
the dependency of the variable Time Between Visits from the variable Time Since Onset.

For the network on the second dataset, the layering was defined as follows.
Layer 1: Sex, Genetics (WT, TARDBP, C9orf72, SOD1, FUS), BMI premorbid
Layer 2: Familiality
Layer 3: Medical centre
Layer 4: Age onset, FTD, Onset site, FVC diagnosis, BMI diagnosis
Layer 5: Diagnostic delay
Layer 6: MITOS, NIV, PEG variables at time (t-1)
Layer 7: Time Between Visits
Layer 8: MITOS, NIV, PEG variables at time t
Layer 9: Survival
Layer 10: Time Since Onset

The following rules were imposed after the training phase:
Layer 1 cannot depend on itself or any other layer.
Layer 2 can only depend on layer 1.
Layer 3 cannot depend on itself or any other layer.
Layer 4 can only depend on itself and layers 1 and 2.
Layer 5 can only depend on layers 1 to 4.
Layer 6 cannot depend on itself or any other layer.
Layer 7 can only depend on layers 3, 6 or 10.
Layer 8 can depend on any other layer, except for itself and layers 7 and 9.
Layer 9 can depend on any other layer, except for itself and layers 7 and 8.
Layer 10 cannot depend on itself or any other layer.

These rules are given in matrix form according to Table 2.

TABLE 2

| | L1 | L2 | L3 | L4 | L5 | L6 | L7 | L8 | L9 | L10 |
|---|---|---|---|---|---|---|---|---|---|---|
| L1 | 0 | 1 | 0 | 1 | 1 | 0 | 0 | 1 | 1 | 0 |
| L2 | 0 | 0 | 0 | 1 | 1 | 0 | 0 | 1 | 1 | 0 |
| L3 | 0 | 0 | 0 | 0 | 1 | 0 | 1 | 1 | 1 | 0 |
| L4 | 0 | 0 | 0 | 1 | 1 | 0 | 0 | 1 | 1 | 0 |
| L5 | 0 | 0 | 0 | 0 | 0 | 0 | 0 | 1 | 1 | 0 |
| L6 | 0 | 0 | 0 | 0 | 0 | 0 | 1 | 1 | 1 | 0 |
| L7 | 0 | 0 | 0 | 0 | 0 | 0 | 0 | 0 | 0 | 0 |
| L8 | 0 | 0 | 0 | 0 | 0 | 0 | 0 | 0 | 0 | 0 |
| L9 | 0 | 0 | 0 | 0 | 0 | 0 | 0 | 0 | 0 | 0 |
| L10 | 0 | 0 | 0 | 0 | 0 | 0 | 1 | 1 | 1 | 0 |

A given element A_{i,j} at row i and column j, if equal to 1 indicates that the variables of layer j can depend on those of layer i. Otherwise, if A_{i,j} is equal to 0, it means that the dependency of layer j on layer i is forbidden.

FIG. 2 reports the network obtained from the second training set.

In both the cases described above, specific sense constraints were thus applied to the network structures to codify the domain knowledge: clinically or biologically nonsensical relations among variables were forbidden, such as, for instance, the dependency of medical centre on patient's sex. As another example, in this second network the dependency of the BMI premorbid from the Time Between Visit was forbidden. As another example, in the case of the Diagnostic Delay, in both the networks, its dependency from any variable recorded after the diagnosis was forbidden.

On the contrary, some other possible relationships were allowed, as, for example, the possible dependency in both networks of the variable Time Between Visits from the Medical Centre, that can have specific protocols in the visit scheduling, and from the values of the MITOS variables at the previous visit, that can influence the visit frequency.

Information relating to the aforesaid first and second datasets will be provided below, used in an implementation example for the training and validation steps of the model used in the present method.

ALS patients were recruited from two population-based registers, in Italy, and four referral ALS centers, two centers in Italy and two centers in Israel.

ALS diagnosis was assessed according to El Escorial revised criteria (Benjamin Rix Brooks, Robert G. Miller, Michael Swash, Theodore L. Munsat “El Escorial revisited: Revised criteria for the diagnosis of amyotrophic lateral sclerosis”. Amyotrophic Lateral Sclerosis and Other Motor Neuron Disorders, 1(5):293{299, 2000. PMID: 11464847).

For each patient, several demographical and clinical characteristics were collected.

The above mentioned first dataset was created by including the information common to all the six Italian and Israeli cohorts, reporting the information collected over subsequent screening visits.

For each patient, the following variables were collected: sex, site of onset (spinal or bulbar), survival (time from ALS onset to either tracheostomy/death, or censoring information, i.e., date of last interaction with the clinical center), age at onset, diagnostic delay (time from ALS onset to diagnosis), and the revised ALS Functional Rating Scale (ALSFRS-R) (Jesse M. Cedarbaum, Nancy Stambler, Errol Malta, Cynthia Fuller, Dana Hilt, Barbara Thurmond, Arline Nakanishi, Bdnf Als Study Group, 1A complete listing of the BDNF Study Group, et al. “The ALSFRS-R: a revised ALS functional rating scale that incorporates assessments of respiratory function”. Journal of the neurological sciences”, 169(1-2):13{21, 1999), which is a 12-item questionnaire rated on a 0-4 point scale evaluating the progression of disability in ALS patients.

The above mentioned second dataset was built by including data only from Italian registers and centres.

In addition to the variables of the first dataset, this second dataset includes: ALS family history, genetics (genes C9orf72, FUS, SOD1 and TARDBP were tested for mutations; if negative, patients were classified as wild type—WT), presence of FTD (detected either clinically or through neuropsychological testing), body mass index (BMI) both premorbid and at diagnosis, FVC at diagnosis, and dates of NIV and percutaneous endoscopic gastrostomy (PEG) procedures, if carried out.

In the exemplary validation activity, here reported, a preprocessing was carried out.

Firstly, both the first and second datasets were filtered by excluding the variables that were missing in more than 50% of the subjects, and by removing all patients with only one visit.

This step resulted in a total of 4026 ALS patients and 24960 data measurements for the first dataset (median follow-up of 27 months, IQR 18-44; median number of visits equal to 5, IQR 3-8), and a total of 2149 ALS patients and 15767 data measurements for the second dataset (median follow-up of 29 months, IQR 19-39; median number of visits equal to 5, IQR 3-9).

Secondly, the ALSFRS-R scores was converted into the well-known “Milano-Torino staging system”, MITOS (according to the algorithm proposed in the scientific paper: Adriano Chia, Edward R. Hammond, Gabriele Mora, Virginio Bonito, Graziella Filippini: “Development and evaluation of a clinical staging system for amyotrophic lateral sclerosis” Journal of Neurology, Neurosurgery & Psychiatry, 86(1):38-44, 2015) obtaining the variables “Breathing, Swallowing, Communicating, Walking/self-care,” referred to the functional impairment domains.

Time between visits (TBV) and time since onset (TSO) were also added, in order to account for different observation-windows and different time-grids among subjects and to explicitly model the variation of the visit frequency as the disease progresses.

Then, each dataset was split into a training set for developing the Dynamic Bayesian Networks, and a test set for validating the model by stratifying the datasets over all variables.

In detail, the first dataset was split into a training set of 3221 and a test set of 805 patients; the second dataset was split into a training set of 1504 and a test set of 645 patients.

Finally, since the developed DBNs encode probabilistic relationships among discrete variables over a discrete number of time steps, continuous variables were discretised according to their distribution percentiles.

Graphs encoding these dependencies and representing the developed DBNs are reported in FIGS. 1 and 2, for the first and the second training set, respectively.

The cycles (autocycles), or loops, on the four variables relating to the functional domains encoded by the MITOS scale (walking/self-care, breathing, swallowing, communicating) in both FIGS. 1 and 2, as well as the cycles (autocycles) on the NIV and PEG variables of FIG. 2, show the dependence of the variable value on the variable value itself at the previous time step.

FIG. 1 reports the network obtained from the first training set. As expected, the values of each MITOS domain at a given time depend on the values of the same domain at the previous time-point (loops).

As defined in the mandatory constraints, time since onset is a parent to all the MITOS domains and survival, in concordance with the progressive nature of the disease over time. The dependency of the time between visits from the MITOS walking/self-care domain indicates the influence of this value recorded during a visit to the following care planning schedule.

Moreover, the model evidenced that the loss of independence in breathing and in communicating at a specific time-point can be predicted by the value of movement in a previous time-point: an impairment in movement increases the probability of experiencing an impairment in communicating and breathing in the next visits.

Furthermore, swallowing and communicating, as well as swallowing and breathing, appear to be inter-related.

The onset site is dependent on both sex (mandatory edge) and age at onset, confirming relationships known in literature: men have a greater likelihood of onset in the spinal regions, while women tend to have higher propensity for bulbar-onset disease; furthermore, bulbar onset is related to higher age at onset.

The survival time depends on time since onset (mandatory edge), age at onset, medical centre and respiratory functionality (breathing). The dependence of survival from both time since onset and breathing is quite intuitive; the dependence from age at onset is already known in the literature, being a longer survival in younger patients probably correlated to their greater neuronal reserve.

The role of the medical centre on survival and, more in general, on the whole network merits closer examination. This variable is also parent to time between visits, indicating a possible different policy in the visit schedule.

The relationship between onset site and swallowing may reflect the direct effect of the bulbar onset on the deglutition ability, with anticipated dysarthria and dysphagia occurrence.

Also, the direct edge from onset site to diagnostic delay validates some results reported in literature. Conversely, in other results reported in literature, a significant difference in the diagnostic delay between bulbar- and spinal-onset patients is not found, leaving this relationship as an open-question.

In the model, the diagnostic delay depends also on sex and age at onset.

Expected relationships among variables can also be found as indirect dependencies. For instance, the linkage between onset site and survival can be identified from the following path in the graph: onset site→swallowing→breathing→survival.

Also, the effect of the diagnostic delay on the survival can be found through the indirect path: diagnostic delay→walking/self-care→breathing→survival.

The graph obtained on the second training test (FIG. 2) is constituted by a higher number of nodes than the graph illustrated in FIG. 1.

As resulting in both the graphs for the MITOS domain impairments, also the NIV and PEG nodes present a loop, indicating that the value of these variables at a given time-point depends on its value at the previous time-point.

According to different policies regarding the life-support interventions, as well as the different centre specialisation levels, medical centre results in a composite effect on NIV, PEG, and survival. These relationships have to be read together with the other occurring parents: NIV depends also on breathing and, indirectly through breathing, on FVC at diagnosis (both variables related to the respiratory functionality); PEG depends on BMI at diagnosis and swallowing (related to the initial and progressive impact of the disease on the nutrition ability). Survival is also dependent on FVC at diagnosis, on NIV and on time since onset (mandatory edge).

Moreover, the genetic aetiology of ALS is correctly modelled in the graph, inferring the role on familial ALS of repeat expansion in C9orf72 and mutations in TARDBP and SOD1.

It is also interesting to notice that there is no dependency between familiality and FUS, in line with the fact that the latter is a de novo mutation. The graph also evidences that FTD is related to mutations in TARDBP and C9orf72 repeat expansion, characteristic already previously associated to FTD phenotypes.

The influence of premorbid BMI on ALS familiality emerges, partially supporting dome literature studies, which evidenced a relationship between premorbid BMI and hypothalamus atrophy, a typical ALS signature, in familial ALS patients.

Similarly to the graph of FIG. 1, indirect relationships can also be identified, as the linkage between onset site and survival defined by the path: onset site→walking/self-care→NIV→survival.

An association between SOD1 and age at onset emerges as direct edge, as well as the one between C9orf72 and age at onset: interestingly, the age-related penetrance of gene mutations is currently an open question in the literature.

With regard to the DBN-based simulations, some exemplary details are provided herein below.

Since the CPDs inferred on the training sets encode the most probable value of a variable given the values of its parents at the previous time point, DBNs allow the simulation of ALS progression starting from the data of the patient at a specific visit.

The first recorded contact with the medical centre is set as starting point for the simulations. The simulation requires a fully-known starting set of variables to run, thus the subsets of patients without missing values in their first visit were extracted from the test sets of the first and second datasets. This filtering step reduced the sizes of the test sets to 719 and 263 patients for the first and second datasets, respectively. Again, it was checked that the reduced test sets maintained the same distributions over all variables as the corresponding training sets.

For each patient, starting from his/her first visit, the temporal evolution of the disease was simulated by sampling the CPDs for 40 consecutive visits or until the simulated death or tracheostomy intervention occurred. The simulation sets the time step between two consecutive visits according to the time steps distribution learnt by the DBNs on the training set, accounting for the variability across patients and stages of the disease. The number of simulated visits was set to a relatively high value (40) so that each patient reaches the tracheostomy/death event with high probability. For each visit, the current values of the variables are simulated, in accordance to the values of their parents at the previous time point, by sampling them from the CPDs. Since this process is probabilistic, 100 different simulations of the disease progression were performed for each patient starting from his/her first visit, in order to obtain a statistic on the simulated prognoses: a total of 71.900 and 26.300 simulations were therefore run for the first and second test set subjects, respectively.

Some information about the model validation methods is provided below, according to an implementation option of the present method.

The simulation process allows the validation of the DBNs. By comparing the simulated prognosis for each patient and the true disease progression, it is in fact possible to assess the prediction accuracy of the learnt DBNs.

The concordance between real and simulated progression was quantified by the simulation error, defined as the difference between the percentages of real and simulated patients that have experienced a clinical outcome, set as either MITOS impairment or tracheostomy/death. A low error corresponds to a high concordance between the real and simulated ALS progressions. This metric was computed for each clinical outcome at consequent time points from 12 to 96 months, with a 12-months step, by stopping at 96 months since the percentage of deceased patients exceeded 95% in the following year.

In addition, the Area Under (AU) the Receiver Operating Characteristic (ROC) curve was used to assess the ability of the DBN models to rank subjects based on their risk of MITOS impairment and tracheostomy/death.

For a given clinical outcome, the ROC represents the probability of a patient who has experienced the outcome to be correctly simulated (true positive rate) versus the probability of a patient who has not experienced the outcome to be incorrectly simulated (false positive rate).

The ROC curves were computed at the same time points set for the simulation error. The AU-ROC indicates the probability that a patient who has experienced a certain clinical outcome is assigned a higher risk value by the model than a patient who has not experienced that outcome yet: higher AU-ROC values (in a possible range 0-1) correspond to better simulation performances.

To evaluate the accuracy of the model over time, the integral of the AU-ROC (iAU-ROC) across all the simulated survival time points up to 96 months was finally computed, for each clinical outcome. The iAU-ROC can be interpreted as a global concordance index measuring the probability that subjects with a large predicted risk value have a shorter time to clinical outcome than subjects with a small predicted risk value.

The DBN-based simulator also allows patient cohort stratification, i.e., the identification of variables whose specific ranges of values could be related to the velocity of disease progression or survival. In detail, it was traced how the change in a specific variable affects the survival or the disease course, by simulating ALS progression of population with specific phenotypes at onset and comparing how they differentiate in terms of disease severity as well as survival time.

Finally, the DBNs were also used to determine the mutual dependencies between the variables in terms of conditional probabilities.

In order to assess and validate the prediction capabilities of the developed DBN models, the progression of ALS in the patients was simulated from the first and second test sets and compared the obtained predictions with the real patient data by using the above-mentioned metrics. Starting from the information of the patients' first visits, we simulated the time to impairment in the four MITOS functional domains and the time to tracheostomy/death.

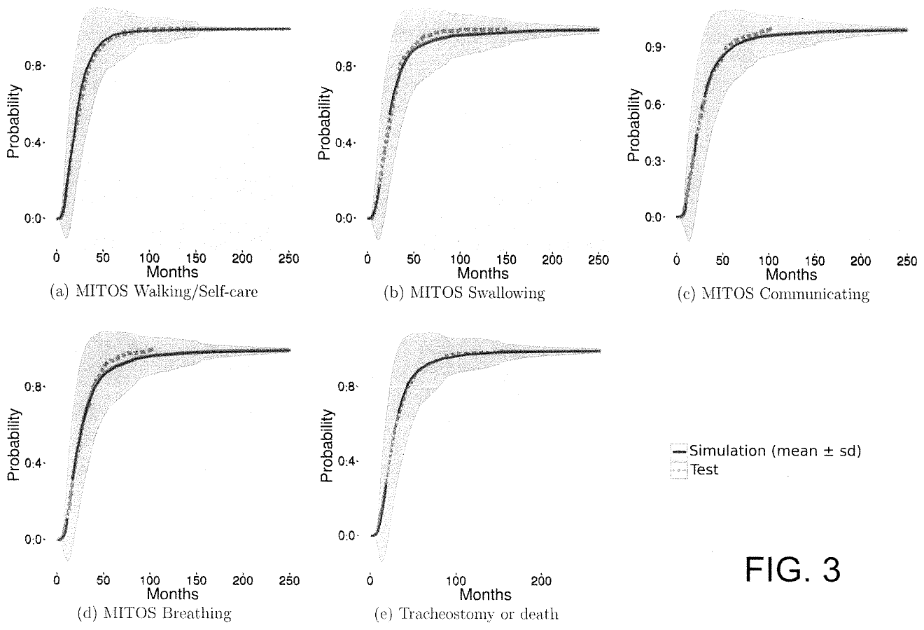

FIG. 3 depicts the cumulative probability of MITOS domain variables impairment and tracheostomy/death over time describing the true (thicker dashed lines) and simulated (thinner continuous lines) ALS progression of the first test set population.

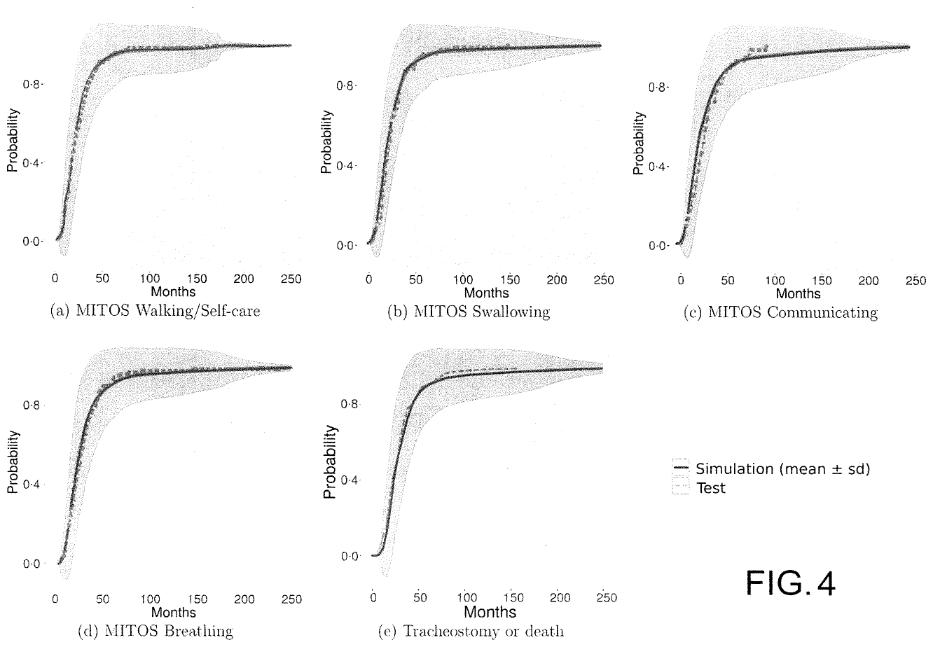

Similarly, FIG. 4 depicts the true and simulated ALS progression of the second test set population.

The figures show a high concordance between the predicted and actual ALS progression for both models, confirming that the DBN models, developed in the present method, provide a precise simulation of survival and MITOS domain impairment.

The time-dependent ROC curves at various time points were computed for each predicted clinical outcome for the patients of the first and second datasets and their AU-ROC values are given in Tables 3 and 4, respectively, reported here below. The last column gives the iAU-ROC values computed over all simulated time points up to 96 months.

TABLE 3

| | AU-ROC at time point [months]: | | | | | | | | |
|---|---|---|---|---|---|---|---|---|---|
| Variables | 12 | 24 | 36 | 48 | 60 | 72 | 84 | 96 | iAU-ROC |
| MITOS Movement | 0.93 | 0.85 | 0.86 | 0.83 | 0.83 | 0.84 | 0.78 | 0.81 | 0.87 |
| MITOS Swallowing | 0.96 | 0.86 | 0.78 | 0.78 | 0.75 | 0.76 | 0.73 | 0.73 | 0.84 |
| MITOS Communication | 0.92 | 0.82 | 0.77 | 0.75 | 0.76 | 0.75 | 0.78 | 0.75 | 0.86 |
| MITOS Breathing | 0.90 | 0.80 | 0.73 | 0.71 | 0.69 | 0.72 | 0.71 | 0.71 | 0.84 |
| Survival time | 0.99 | 0.92 | 0.85 | 0.81 | 0.80 | 0.82 | 0.83 | 0.80 | 0.89 |

TABLE 4

| | AU-ROC at time point [months]: | | | | | | | | |
|---|---|---|---|---|---|---|---|---|---|
| Variables | 12 | 24 | 36 | 48 | 60 | 72 | 84 | 96 | iAU-ROC |
| MITOS Movement | 0.90 | 0.85 | 0.86 | 0.87 | 0.87 | 0.85 | 0.82 | 0.89 | 0.93 |
| MITOS Swallowing | 0.96 | 0.87 | 0.85 | 0.83 | 0.82 | 0.82 | 0.82 | 0.92 | 0.93 |
| MITOS Communication | 0.99 | 0.82 | 0.79 | 0.83 | 0.84 | 0.80 | 0.76 | 0.85 | 0.91 |
| MITOS Breathing | 0.89 | 0.85 | 0.82 | 0.78 | 0.81 | 0.81 | 0.82 | 0.88 | 0.92 |
| Survival time | 0.95 | 0.86 | 0.85 | 0.83 | 0.81 | 0.83 | 0.89 | 0.93 | 0.93 |

The AU-ROC values obtained by the first dataset model range from 0.69 to 0.96 for the impairment prediction in the four MITOS domains, and from 0.80 to 0.99 for the prediction of survival time. The iAU-ROC range from 0.84 to 0.89, denoting a good concordance of the predictions with the actual ALS evolution.

The second dataset model obtained AU-ROC values ranging from 0.76 to 0.99 for the impairment prediction in the four MITOS domains, and from 0.81 to 0.95 for the prediction of survival time. The iAU-ROC range from 0.91 to 0.93, denoting a very good concordance of the predictions with the actual disease progression. The results on both the DBNs confirm the ability of the models to simulate clinically reliable ALS population by using the first screening visit only.

It is worth noticing that the model developed on the second dataset, although trained on a smaller number of patients, obtained overall better predictions than its counterpart built on the first dataset. This is most likely due to fact that the second model contains more variables and can thus better capture the ALS progression mechanisms.

A method for determining a statistical classification and/or stratification of patients suffering from ALS, carried out by electronic processing and/or calculating means, which is also comprised in the present invention, is described below.

Such method comprises the steps of carrying out a method for determining a disease progression and survival prognosis for patients suffering from amyotrophic lateral sclerosis, according to any of the previously described embodiments, on each patient of a plurality of patients; and processing the plurality of respective results obtained to determine a statistical classification and/or stratification in subgroups with specific clinical manifestations and prognosis.

According to another aspect, the present invention comprises a method for identifying and/or weighing risk factors of amyotrophic lateral sclerosis (ALS), carried out by electronic processing and/or calculating means.

Such method firstly comprises a step of defining a set of variables associated with the onset, progression of amyotrophic lateral sclerosis, in which such set of variables comprises a first group of variables associated with the onset of amyotrophic lateral sclerosis, comprising at least the variables “patient sex”, “disease onset age”, “disease onset site”; a second group of temporal variables comprising at least the variables “time elapsed since disease onset”; a third group of dynamic functional variables associated with disease effects, comprising at least one of the variables breathing, swallowing, communicating, movement or at least one variable of a functional progression and/or severity scale of amyotrophic lateral sclerosis; and at least one variable associated with survival.

The method further comprises the further steps of encoding by means of a Dynamic Bayesian Network, using at least one trained algorithm, a plurality of probabilistic conditional dependence relationships, in which each relationship is a probabilistic conditional dependence relationship between two of said variables; then, defining the prediction times, so that each prediction time belongs to a respective time interval in which the conditional dependence relationships between the variables are stationary, that is, time-invariant or homogeneous; then, defining a temporal variable representative of the prediction time.

The method further involves describing the aforementioned Bayesian Dynamic Network, using at least one trained algorithm, by means of a corresponding graph, comprising the aforesaid variables as nodes and comprising topological connections oriented between nodes corresponding to variables among which a probabilistic conditional dependence is identified.

In such graph, given a node, the connections entering therein represent a conditional probability of the value assumed by the variable associated with the node depending on the values of the variables associated with the nodes from which such connections originate. At least one of such connections is associated with a conditional probability of the value of the variable in which the connection is entering, in a given prediction time, depending on the value of the variable from which the connection is leaving in a previous prediction time.

Furthermore, for the nodes associated with the functional dynamic variables belonging to the third group of variables, respective local cycle connections entering and leaving the same node are expected, adapted to describe the influence of the respective dynamic functional variable on itself over time.

The method further comprises the steps of entering, for each of the defined variables, data acquired at a given acquisition time relating to the situation of a specific patient; and calculating, by electronic processing and/or calculating means, on the basis of the aforesaid Dynamic Bayesian Network and the aforesaid graph, and starting from the aforesaid acquired data, the values of each of the defined variables, at one or more prediction times following the acquisition time.

Finally, the method involves identifying and/or weighing risk factors of amyotrophic lateral sclerosis (ALS) on the basis of said graph and the calculated values of such variables.

As noted above, the DBN models developed and used in the method of the present invention can be used both for analysis on entire populations and for probabilistically predicting the disease progression of a single patient with ALS, on the basis of information recorded during a specific visit of the patient.

The disease temporal evolution of the patient is simulated starting from the recorded values of the variables by sampling the CPDs for a certain number of steps in accordance to the state at the previous time point. The simulation for a given patient is run several times in order to obtain an estimate of the probability of occurrence probability of the outcome of interest.

In accordance with an embodiment, the method comprises the further step of providing a computerized graphical interface, configured to receive input data relating to patient variable values, relating to a specific instant in time, and to display the temporal evolution prediction results of the third group and/or survival prediction variables.

According to an implementation option, the computerized graphical interface comprises a “dashboard” made available to medical or clinical personnel in the form of an interactive web application, which shows a prognostic prediction for a single patient.

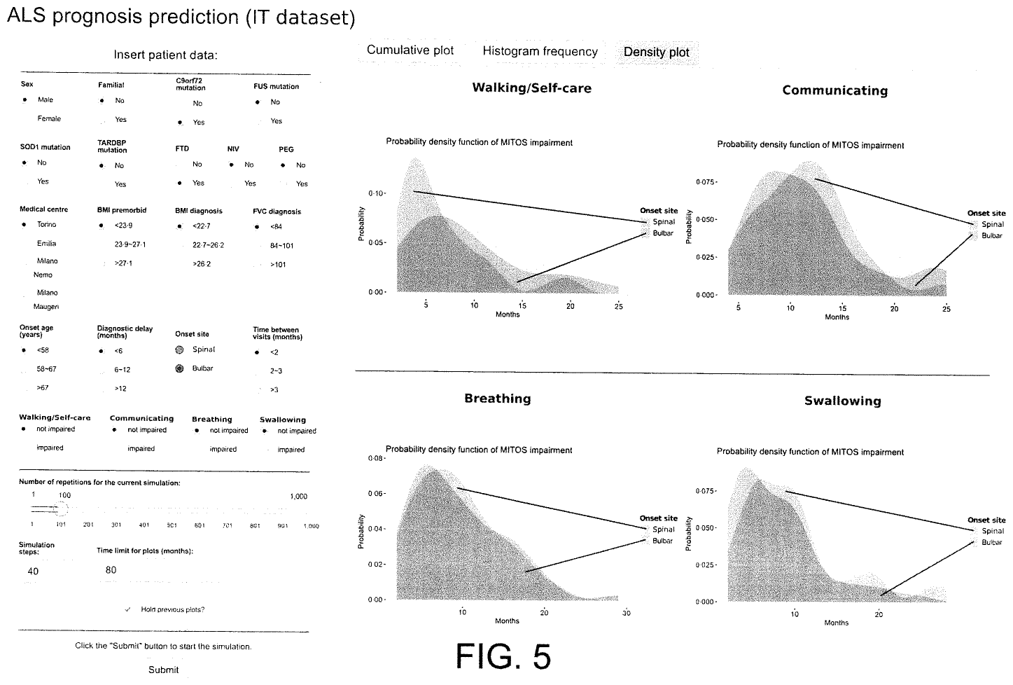

FIG. 5 shows an exemplary GUI of the above mentioned web application. The physician can enter the clinical data recorded during the first contact with the patient in the left side of the screen, under the “Insert patient data” label, and then start the simulation with up to 1000 repetitions (100 repetitions were used in the presented example). The plots on the right side of the screen give the probability of impairment in each of the four main MITOS domains.

According to an implementation option, different simulations can be run sequentially, allowing the user to decide whether to keep the plots from previous simulations to be viewed alongside with the plots from the last one. This way, it is possible to estimate the effect of one or more biomarkers on the ALS prognosis: for instance, FIG. 5 compares the effects of the “spinal” and “bulbar” onsets while leaving all other parameters unchanged.

Similarly, it is possible to simulate an untreated population, which could serve as control group for clinical trials.

In this sense, the dashboard can be used to generate in silico populations.

In summary, a probabilistic predictor of the progression of ALS has been developed by building DBN models on the data contained in six datasets: two from population-based ALS registries and four from referral ALS centres, from Italy and Israel. Being comprised of patient visits from clinical contexts and partially never investigated before, the datasets employed in this work are more representatives of the general ALS population than clinical trial databases, as the PRO-ACT dataset.

Trained with the entire dynamic of the available data of disease progression, models developed with the present method can be used to simulate and/or to predict, starting from a single time point, the entire disease progression in terms of time to the loss of independence in walking/self-care, swallowing, communication and breathing and time to death.

The prediction accuracy was assessed by comparing the predicted patients' prognoses with the real data: different performance metrics confirmed that the proposed models possess good performance in terms of both survival and domain impairment prediction.

The method can also be used to stratify ALS patients into subgroups of different progression and to assess the effect of single phenotypes at diagnosis on the entire disease course.

Relying on DBNs, the present method allows the identification and explicit representation of the relationships between the different variables and of the pathways along which they influence the disease evolution.

Several notable inter-dependencies among variables were identified and validated by comparison with literature results.

Given a specific variable, its parents in the DBN graph can be intended as “composite biomarkers”, since the value of the variable at a certain time point can be inferred by the values of the parents at the previous one, thus extending the classic “standalone” biomarkers that have been used to date.

As can be seen, the objects of the present invention, as indicated above, are fully achieved by the method described above, by virtue of the features illustrated in detail above.

In fact, the method comprises a Dynamic Bayesian Networks (DBNs) based model of ALS progression able to predict and simulate, in a probabilistic fashion, the evolution of ALS over time, thus providing an explicit representation of the temporal nature of the medical problem in terms of changes/loss of independence in the most relevant functional domains impaired by the disease, such as walking/self-care, swallowing, communicating and breathing, besides survival.

Furthermore, the method allows an accurate representation of the domain knowledge and describe the dynamics of the ALS course also in terms of interactions among variables both within and across different points in time, unveiling their impact on disease progression.

Notably, the method includes a methodological novelty to account for the fact that variable dependencies might vary over time, due to the long term evolution of the disease.

Moreover, depending on the information available, two different sub-models have been developed, integrating data from different datasets (as illustrated above). The first sub-model is based on the more frequently available prognostic variables, such as sex, onset site, age at onset, diagnostic delay and the revised ALS Functional Rating Scale; the second one additionally includes features recognized as potentially prognostic in the scientific literature, such as genetic predictors, ALS family history, presence of FTD, body mass index (BMI) premorbid and at diagnosis, premorbid FVC, and the administration of respiratory and nutritional support interventions.

The method can be executed through an interactive web application that can be used by the clinicians to simulate the most probable prognosis of a patient already at his/her first visit. An instrument able to simulate patients' outcomes in the main areas of disability can have a strong and advantageous impact in scheduling the allocation of the resources both at individual and health system level, likely reducing the cost of the care by improving the provision of pharmacological and non-pharmacological therapies.

To the embodiments of the method described above, a person skilled in the art may, in order to meet contingent needs, make modifications, adaptations and substitutions of elements with other functionally equivalent ones without departing from the scope of the following claims. Each of the features described as belonging to a possible embodiment may be implemented independently of the other described embodiments.

1. A method for determining a disease progression and survival prognosis, at a succession of prediction times, for patients suffering from amyotrophic lateral sclerosis (ALS), wherein the method comprises:
defining a set of variables associated with the onset and progression of amyotrophic lateral sclerosis, comprising:
a first group of variables associated with the onset of amyotrophic lateral sclerosis comprising at least the variables “patient sex,” “disease onset age,” “disease onset site;”
a second group of dynamic time variables comprising at least the variable “time elapsed since disease onset”;
a third group of dynamic functional variables associated with disease effects comprising at least one of the variables breathing, swallowing, communicating, walking/self-care or at least one variable of a functional amyotrophic lateral sclerosis progression and/or severity scale;
at least one variable associated with survival;
wherein the method comprises the further steps of:
encoding by means of a Dynamic Bayesian Network, using at least one trained algorithm, a plurality of probabilistic conditional dependence relationships, wherein each relationship is a probabilistic conditional dependence relationship between two of said variables;
defining said prediction times, so that each prediction time belongs to a respective time interval wherein the conditional dependence relationships between the variables are stationary, that is, time-invariant or homogeneous;
defining a time variable representative of the prediction time;
describing said Dynamic Bayesian Network, using at least one trained algorithm, by means of a corresponding graph, comprising said variables as nodes and comprising topological connections oriented between nodes corresponding to variables among which a probabilistic conditional dependence is identified;
wherein, given a node, the connections entering therein represent a conditional probability of the value assumed by the variable associated with said node depending on the values of the variables associated with the nodes from which such connections originate;
wherein at least one of said connections is associated with a conditional probability of the value of the variable in which the connection is entering, in a given prediction time, depending on the value of the variable from which the connection is leaving in a previous prediction time;
and wherein, for the nodes associated with the dynamic functional variables belonging to the third group of variables, respective local cycle or local loop connections entering and leaving the same node are expected, adapted to describe the influence of the respective dynamic functional variable on itself over time;
entering, for each of the defined variables, data acquired at a given acquisition time, relating to the situation of a specific patient;
calculating, by electronic processing and/or calculating means, on the basis of said Dynamic Bayesian Network and said graph, and starting from said acquired data, the values of each of the defined variables, at one or more prediction times following the acquisition time;
obtaining disease progression prognosis results, at a given prediction time, on the basis of the values of one or more of the third group variables calculated at said prediction time;
obtaining survival prognosis results at a given prediction time, on the basis of the value of at least one variable associated with survival, calculated at said prediction time.

2. A method according to claim 1, wherein the set of variables only comprises said first group of variables associated with the onset of amyotrophic lateral, second group of dynamic time variables, third group of dynamic functional variables and a fourth group of variables comprising at least said variable associated with survival.

3. A method according to claim 1, wherein the set of variables comprises a fifth set of variables comprising genetic variables representing the presence of possible “genetic mutations.”

4. A method according to claim 3, wherein said fifth group of variables comprises the variables: WT, C9orf72, TARDBP, SOD1, FUS.

5. A method according to claim 1, wherein said first group of variables associated with the onset of amyotrophic lateral sclerosis further comprises one or more of the following variables: presence of “frontotemporal dementia (FTD)” and/or “body mass index (BMI) prior to disease onset,” and/or “diagnostic delay” and/or “medical center following the patient” and/or “familiality,” and/or “body mass index (BMI) at diagnosis” and/or “forced vital capacity (FVC).”

6. A method according to claim 1, wherein said second group of dynamic time variables further comprises the variable “time between consecutive visits.”

7. A method according to claim 1, wherein said third group of dynamic functional variables comprises all the variables breathing, swallowing, communicating, walking/self-care.

8. A method according to claim 7, wherein said third group of dynamic functional variables further comprises “non-invasive ventilation (NIV)” and “percutaneous endoscopic gastrostomy (PEG).”

9. A method according to claim 1, wherein the third group of dynamic functional variables comprises at least one variable of an ALSFRS-R functional scale.

10. A method according to claim 1, wherein each connection of the graph is associated with a conditional probability of the value of the variable in which the connection is entering, in a given prediction time, depending on the value of the variable from which the connection is leaving in a previous prediction time.

11. A method according to claim 1, wherein said graph is a direct graph.

12. A method according to claim 1, wherein said step of describing the Dynamic Bayesian Network, by means of a corresponding graph, using at least one trained algorithm, is carried out in a preliminary training step comprising the steps of:
i) inference of the topology of the graph and
ii) learning the parameters of each conditional probability distribution (CPD), corresponding to the probability that a variable assumes a specific conditional value on each possible joint assignment of values, that is, on the possible combinations of values, of the variables in the parent nodes thereof.

13. A method according to claim 12, wherein said preliminary training step is carried out on the basis of one or more available experimental datasets, divided into a training set and a test set, on which machine learning and/or data mining algorithms are applied.

14. A method according to claim 13, wherein the training step is carried out by dividing the population pathology evolution time interval into sub-intervals, within which lies the temporal stationarity hypothesis of the relationships for the dynamic functional variables of the third group and the time variable of the second group, “time elapsed since disease onset”.

15. A method according to claim 1, wherein said step of calculating the values of each of the variables defined at one or more successive times comprises iterating the following procedure:
calculating the value of each of the variables corresponding to the nodes of the graph in an instant t+1, or predictive time t+1, on the basis of the values of the variables associated with the respective parent nodes at the instant t, or predictive time t, sampling according to the probability values obtained from the conditional probability distribution inferred for the graph.

16. A method according to claim 1, wherein said step of obtaining disease progression prognosis results comprises:
predicting a temporal evolution of the dynamic functional variables of the third group.

17. A method according to claim 1, further comprising a step of supplying and/or making available and/or displaying digital data corresponding to the prognosis and/or survival prediction results.

18. A method according to claim 1, comprising the further step of:
providing a computerized graphical interface, configured to receive input data relating to patient variable values, relating to a specific instant in time, and to display the temporal evolution prediction results of the third group and/or survival prediction variables.

19. A method for determining a statistical classification and/or stratification of patients suffering from ALS, carried out by electronic processing and/or calculating means, comprising the steps of:
carrying out a method for determining a disease progression and survival prognosis for patients suffering from amyotrophic lateral sclerosis, according to claim 1, on each patient of a plurality of patients;
processing the plurality of respective results obtained to determine a statistical classification and/or stratification in subgroups with specific clinical manifestations and prognosis.

20. A method for identifying and/or weighing risk factors of amyotrophic lateral sclerosis (ALS), carried out by electronic processing and/or calculating means, comprising the steps of:
defining a set of variables associated with onset and progression of amyotrophic lateral sclerosis, wherein said set of variables comprises:
a first group of variables associated with the onset of amyotrophic lateral sclerosis comprising at least the variables “patient sex,” “disease onset age,” “disease onset site;”
a second group of time variables comprising at least the variable “time elapsed since disease onset”;
a third group of dynamic functional variables associated with disease effects comprising at least one of the variables breathing, swallowing, communicating, walking/self-care or at least one variable of a functional amyotrophic lateral sclerosis progression and/or severity scale;
at least one variable associated with survival;
wherein the method comprises the further steps of:
encoding by means of a Dynamic Bayesian Network, using at least one trained algorithm, a plurality of probabilistic conditional dependence relationships, wherein each relationship is a probabilistic conditional dependence relationship between two of said variables;
defining said prediction times, so that each prediction time belongs to a respective time interval wherein the conditional dependence relationships between the variables are stationary, that is, time-invariant or homogeneous;
defining a time variable representative of the prediction time;
describing said Dynamic Bayesian Network, using at least one trained algorithm, by means of a corresponding graph, comprising said variables as nodes and comprising topological connections oriented between nodes corresponding to variables among which a probabilistic conditional dependence is identified;
wherein, given a node, the connections entering therein represent a conditional probability of the value assumed by the variable associated with said node depending on the values of the variables associated with the nodes from which such connections originate;
wherein at least one of said connections is associated with a conditional probability of the value of the variable in which the connection is entering, in a given prediction time, depending on the value of the variable from which the connection is leaving in a previous prediction time;
and wherein, for the nodes associated with the dynamic functional variables belonging to the third group of variables, respective local cycle connections entering and leaving the same node are expected, adapted to describe the influence of the respective dynamic functional variable on itself over time;
entering, for each of the defined variables, data acquired at a given acquisition time, relating to the situation of a specific patient;
calculating, by electronic processing and/or calculating means, on the basis of said Dynamic Bayesian Network and said graph, and starting from said acquired data, the values of each of the defined variables, at one or more prediction times following the acquisition time;
identifying and/or weighing risk factors of amyotrophic lateral sclerosis (ALS) on the basis of said graph and the calculated values of said variables.
